(19) United States
(12) Patent Application Publication
(10) Pub. No.: US 2024/0185963 A1
(43) Pub. Date: Jun. 6, 2024

(54) PROTEIN KINETIC ENSEMBLE PLATFORM FOR DISCOVERY OF BINDING POCKETS AND NOVEL LIGANDS

(71) Applicant: ENSEM THERAPEUTICS, INC., Waltham, MA (US)

(72) Inventor: Minghong Hao, Waltham, MA (US)

(21) Appl. No.: 18/528,393

(22) Filed: Dec. 4, 2023

Related U.S. Application Data
(60) Provisional application No. 63/429,654, filed on Dec. 2, 2022.

(51) Int. Cl. G16C 20/50

(52) U.S. Cl. CPC G16C 20/50

(57) ABSTRACT

The invention provides a kinetic ensemble system for identifying non-obvious target pockets and/or generating lead compound structures and/or structural components, comprising computational components and experimental components or processes, wherein the computational components comprise: physics-based simulation and artificial intelligence; and the experimental components or processes comprise biophysical components and chemical biology components.

### PRIORITY CLAIMS AND RELATED APPLICATIONS

This application claims the benefit of priority to U.S. Provisional Application No. 63/429,654, filed Dec. 2, 2022, the entire content of which is incorporated herein by reference for all purposes.

### BACKGROUND OF THE INVENTION

Traditional drug discovery is both a time consuming and expensive process, with a high rate of attrition from the lead discovery to pre-clinical stage and throughout to clinical studies. One of the critical challenges in drug development is the identification and validation of suitable drug targets or binding pockets.

Artificial intelligence (AI) and machine learning (ML), a branch of computer science, statistics, and engineering, utilizes algorithms or models to perform tasks and exhibit behaviors such as learning, making decisions, and making predictions. ML, a subset of AI, allows models to be developed by training algorithms through analysis of data. AI/ML algorithms can be used to analyze genetic, genomic, and proteomic data to pinpoint potential disease targets.

There is an ongoing need to develop AI/ML methodologies and tools that can help researchers to identify and evaluate druggable pockets, to add in drug design and prediction of potency and specificity, and to assist researchers to make informed decisions.

### SUMMARY OF THE INVENTION

In one aspect, the invention relates to a kinetic ensemble system for identifying non- obvious target pockets and/or generating lead compound structures and/or structural components, comprising computational components and experimental components or processes, wherein the computational components comprise: physics-based simulation and artificial intelligence; and the experimental components or processes comprise biophysical components and chemical biology components.

### BRIEF DESCRIPTION OF THE DRAWING

FIG. 1 shows a schematic of an exemplary AI/ML transfer learning enabled lead generation.

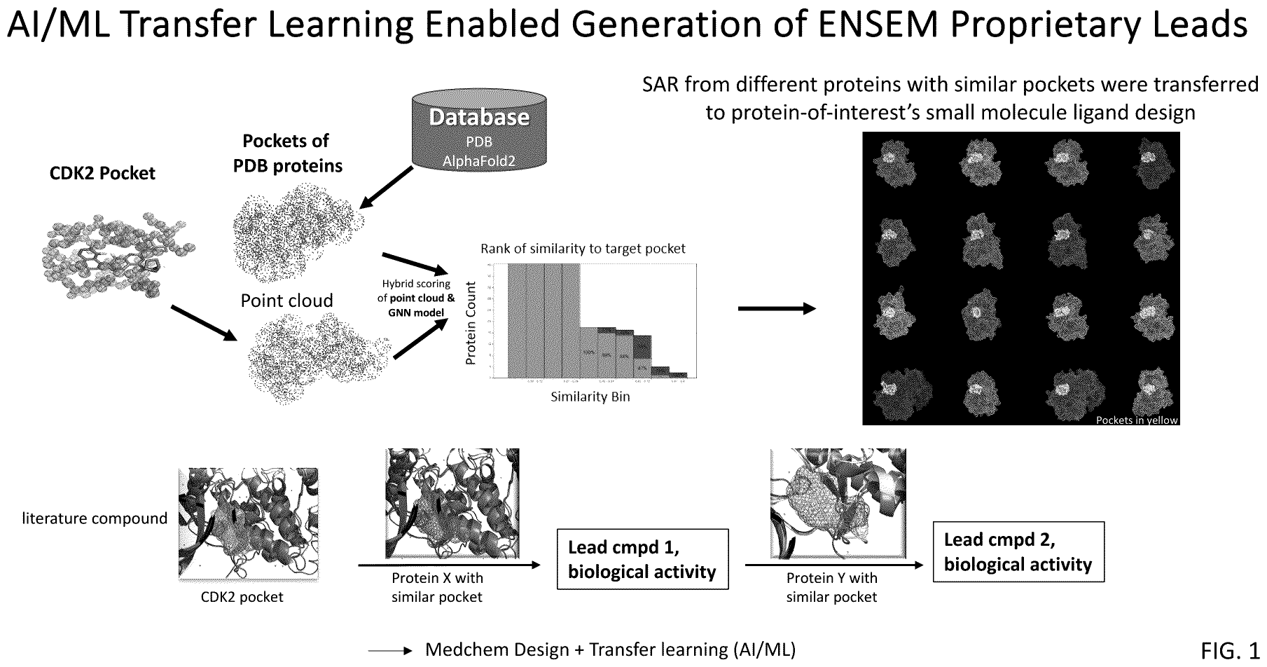

FIG. 2 shows an exemplary study on target “non-obvious” pockets revealed from protein confirmational dynamics and pockets, based on multi-scale MD simulation.

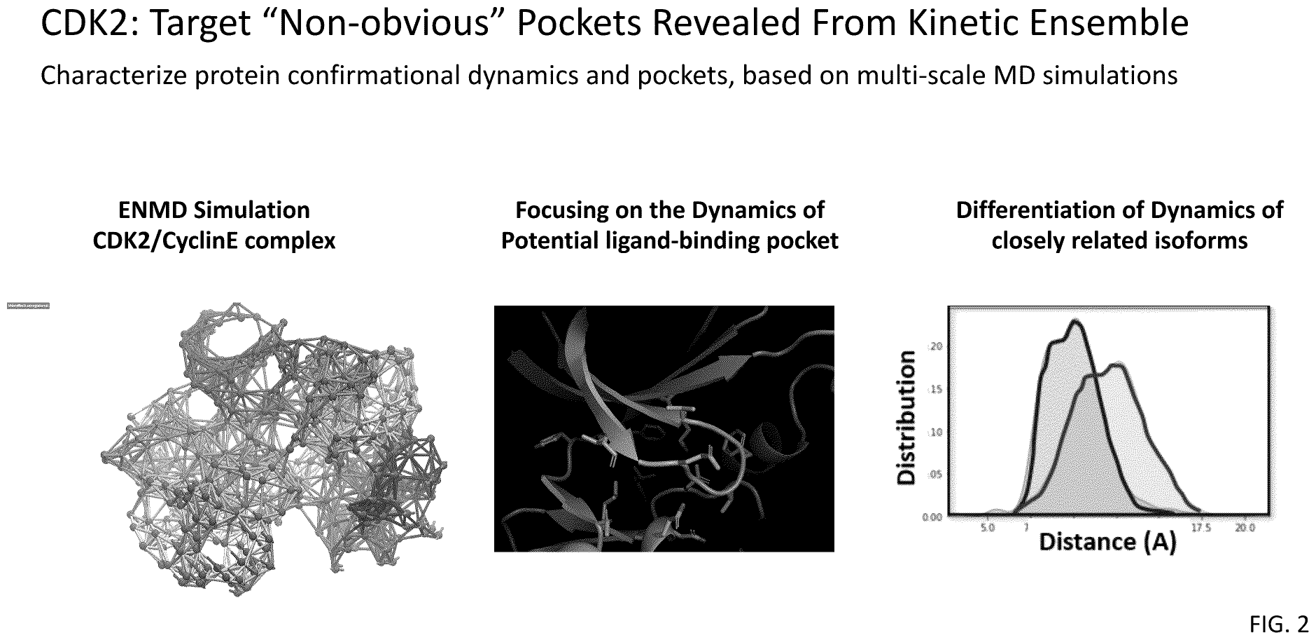

FIG. 3 shows an exemplary study on target “non-obvious” pockets revealed from computational approaches to identify allosteric pocket.

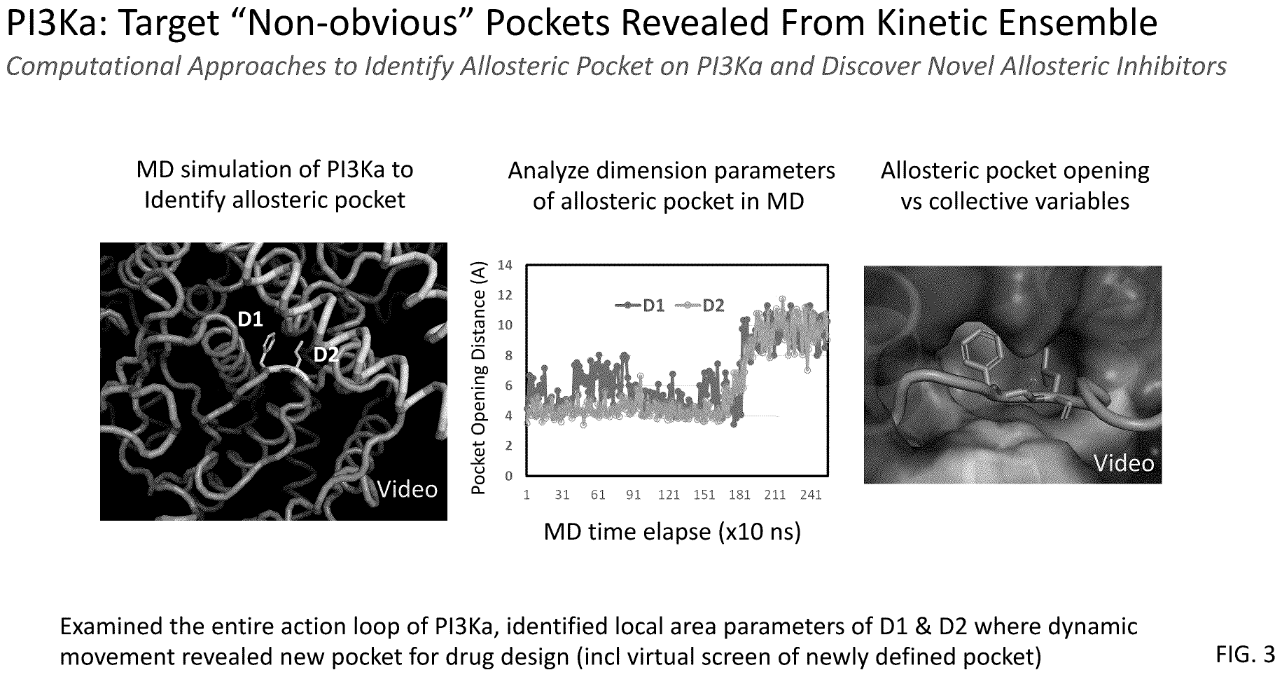

FIG. 4 shows a schematic of exemplary components of the kinetic ensemble platform.

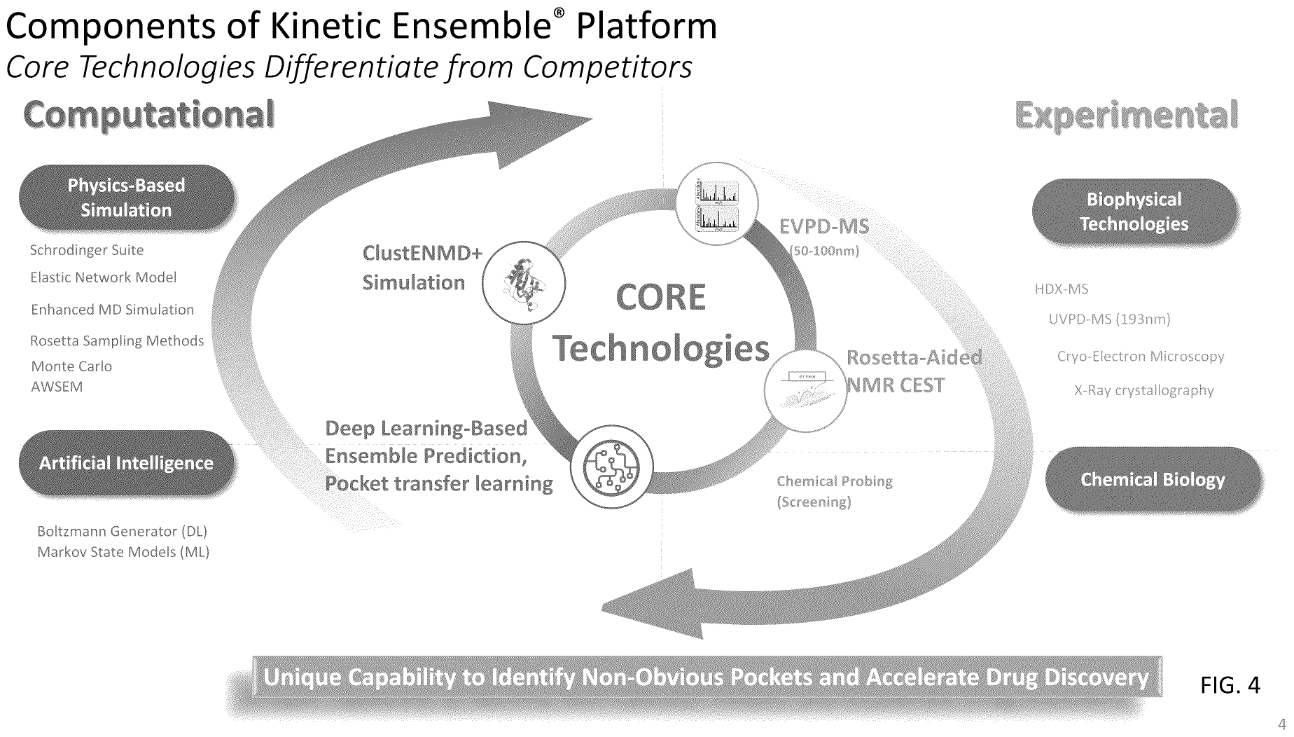

### DETAILED DESCRIPTION OF THE INVENTION

The Kinetic Ensemble platform disclosed herein includes a combination of computational technology and experimental studies or processes. The computational technology includes 1) physics-based simulation and 2) artificial intelligence. The experimental components include biophysical technologies and chemical biology.

The physics-based simulation can be carried out using one or more available programs such as Schrödinger Suite, Elastic Network Model (ENM), Enhanced MD Simulation, Rosetta Sampling Methods (e.g., “RosettaES: a sampling strategy enabling automated interpretation of difficult cryo-EM maps,”Nature Methods, 14, 797-800 (2017)) or Monte Carlo AWSEM (Monte Carlo refers to a mathematical technique that predicts possible outcomes of an uncertain event. AWSEM refers to associative memory, water mediated, structure and energy model). ENMs include entropic models that have demonstrated in many previous studies their abilities to capture overall the important internal motions, with comparisons having been made against crystallographic B-factors and NMR conformational variabilities.

Specific simulation can utilize ClustENMD+Simulation, a combination of ENM, MD or enhanced MD simulation, and clustering which includes a computational intelligence technique for dividing MD conformations into structurally homogeneous groups and for quickly understanding the resulting sets.

The artificial intelligence (AI) component may include Boltzmann Generator (DL) or Markov State Models (ML). Detailed description of these methodologies can be found in, e.g., “Boltzmann generators: Sampling equilibrium states of many-body systems with deep learning,” ScienceVol. 365, Issue 6457 (2019); “Markov State Models to Study the Functional Dynamics of Proteins in the Wake of Machine Learning,”JACS Au2021, 1, 9, 1330-1341. Specific AI component may involve Deep Learning-Based Ensemble Prediction or Pocket transfer learning.

Biophysical technologies include one or more of HDX-MS: Hydrogen Deuterium Exchange Mass Spectrometry, UVPD-MS: UltraViolet-Photodissociation-Mass Spectrometry, cryo-electron microscopy, or X-ray crystallography. Further selected biophysical technology includes EVPD-MS: Extreme UltraViolet-Photodissociation-Mass Spectrometry or Rosetta- Aided NMR Chemical Exchange Saturation Transfer (CEST).

Using the Kinetic Ensemble platform, high quality development candidates have been obtained. For example: new pockets from a target protein were revealed from Kinetic Ensemble by characterizing protein confirmational dynamics and pockets, based on multi-scale MD simulations; further, PDB-wide pocket similarity screening and SAR transfer learning enabled generation of new compounds based on known ones. AI-based potency and DMPK predictions further accelerated the delivery of Development Candidate-quality compounds.

### EXAMPLES

### Kinetic Ensemble®-driven High-Quality CDK2 Lead Compounds:

Novel Lead Compounds Show Superiority Over Benchmark Compounds in the Literature:

1. Optimal combination of hydrophobic and H-bond interactions in a new substituent-induced pocket of CDK2 protein

2. Improved biochemical and biology profiles in vitro

3. 5-10 x more potent than PF-07104091, with excellent concordance of PD biomarker and cell proliferation

4. 100′s fold selectivity over other isoforms of CDKs

5. Broad ScanMax KinomeScan and SpectrumScreen (off-target panel, by Eurofins)

6. Dose-dependent & well tolerated anti-tumor efficacy in vivo via oral administration in tumor-bearing mice

7. Desirable DMPK and safety profiles

8. Higher potency combined with higher bioavailability endows the potential of a better clinical compound than benchmark.

### Novel Allosteric Inhibitors:

Pan Mutant PI3Kαmutants (E542X, E545X, H1047X) or H1047R-specific allosteric inhibitors:

1. Mutant selective PI3Ka inhibitor

2. Biochemical and cellular potency (incl pAKT as cellular biomarker) are comparable to that of benchmark compound

3. Cellular selectivity is superior to benchmark: cell growth inhibition IC50 ratio of WT/Mut PI3Kαcell lines

4. Lead compound with desirable DMPK profiles, with superiority over benchmark

5. Improved aqueous solubility, which may result in improved oral bioavailability.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

The described features, structures, or characteristics of Applicant's disclosure may be combined in any suitable manner in one or more embodiments. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A kinetic ensemble system for identifying non-obvious target pockets and/or generating lead compound structures and/or structural components, comprising computational components and experimental components or processes, wherein the computational components comprise: physics-based simulation and artificial intelligence; and the experimental components or processes comprise biophysical components and chemical biology components.
